(19) United States
(12) Patent Application Publication
(10) Pub. No.: US 2014/0261429 A1
(43) Pub. Date: Sep. 18, 2014

(54) OXYGEN-SUPPLYING RESPIRATOR REQUIRING NO ELECTRIC POWER

(71) Applicant: Chi-Sheng TSAI, Taipei City (TW)

(72) Inventor: Chi-Sheng TSAI, Taipei City (TW)

(21) Appl. No.: 13/798,118

(22) Filed: Mar. 13, 2013

(51) Int. Cl. A61M 16/20; A61G 10/02; A61M 16/00

(52) U.S. Cl. CPC A61M 16/20; A61M 16/0078; A61G 10/026
USPC 128/205.13; 128/205.24

(57) ABSTRACT

An oxygen-supplying respirator requiring no electric power is provided. The oxygen-supplying respirator, which is driven not by electricity but by the oxygen supply pressure at an external oxygen supply end, includes an oxygen supply valve unit in communication with the oxygen supply end so as to turn on and off oxygen output therefrom. An inhalation/exhalation time setting valve unit is in communication between the oxygen supply valve unit and the oxygen supply end, receives the oxygen continuously supplied from the oxygen supply end, and outputs oxygen to the oxygen supply valve unit intermittently. Thus, the oxygen supply valve unit is driven to turn on the oxygen supply end intermittently and thereby enable intermittent oxygen output. The respirator can supply oxygen to a patient where there is no electricity.

### BACKGROUND OF THE INVENTION

1. Technical Field

The present invention provides an oxygen-supplying respirator requiring no electric power. More particularly, the present invention relates to an oxygen-supplying respirator whose operation is driven not by electricity but by the oxygen supply pressure at an oxygen supply end.

2. Description of Related Art

Nowadays, respirators for use in hospitals are typically of the volume-assured pressure support (VAPS) ventilation mode, which is a combination of pressure support ventilation and volume control ventilation, and in which the inhalation pressure reaches a predetermined value each time a user initiates a respiratory action. Such a respirator can monitor a patient's tidal volume (Vt) so that pressure-targeted ventilation can be achieved with the tidal volume guaranteed for each respiratory action.

Respirators of this mode not only are applicable to the acute respiratory distress syndrome, but also are helpful in enabling patients to terminate the use of respirators during recuperation. This is because the VAPS ventilation mode reduces the physical work required for breathing and allows a patient's body to function in concert with the respirator.

As the conventional VAPS ventilation respirators are electricity-driven, they are used mainly in the general wards, intensive care units, or emergency room of a hospital or in ambulances. However, the need to use such respirators is by no means limited to hospitals and ambulances but exists wherever disasters may take place. More importantly, power outage is very likely to occur where disaster rescue operations are conducted, a notable example of which is what happened in the wake of the tsunami devastating Japan on Mar. 11, 2011. Where there is no electric power, the conventional respirators cannot be turned on, let alone saving people's lives in time. Hence, the conventional respirators need improvement.

### BRIEF SUMMARY OF THE INVENTION

The primary object of the present invention is to provide an oxygen-supplying respirator which is driven not by electricity but by the oxygen supply pressure at an oxygen supply end and which, therefore, overcomes the aforesaid drawback of the prior art respirators, namely the dependency on electricity to operate. As stated above, such dependency on electric power renders the conventional respirators useless in the lack of electricity, e.g., in disaster rescue operations carried out away from hospitals and ambulances.

To achieve the above object, the present invention provides an oxygen-supplying respirator requiring no electricity, wherein the oxygen-supplying respirator includes an oxygen supply valve unit in communication with an external oxygen supply end so as to turn on and off oxygen output from the oxygen supply end. The oxygen-supplying respirator is characterized in the following:

An inhalation/exhalation time setting valve unit is in communication between the oxygen supply valve unit and the oxygen supply end. The inhalation/exhalation time setting valve unit receives the oxygen continuously supplied from the oxygen supply end and outputs oxygen intermittently to the oxygen supply valve unit, thereby driving the oxygen supply valve unit to turn on the oxygen supply end intermittently to enable intermittent oxygen output.

In use, an oxygen (or pressurized mixed-gas or pressurized air) supply end can be used to supply oxygen continuously to the inhalation/exhalation time setting valve unit and the oxygen supply valve unit, thereby driving the inhalation/exhalation time setting valve unit to output oxygen intermittently to the oxygen supply valve unit. The oxygen supply valve unit is driven by the intermittently output oxygen to turn on the oxygen supply end intermittently, allowing oxygen (or a pressurized mixed gas or pressurized air, hereinafter collectively referred to as oxygen) to be intermittently output to a patient.

Thus, the object of driving the respirator with the oxygen supply pressure at an oxygen supply end rather than with electricity is attained.

More specifically, the present invention may further include the following features:

In addition to the main structural features described above, the oxygen supply valve unit includes a pilot-operated cylinder and a gas-operated valve. The gas-operated valve is driven by the pilot-operated cylinder to turn on or off oxygen output from the oxygen supply end to a patient. Moreover, the inhalation/exhalation time setting valve unit includes a timing valve module. The timing valve module has an exhalation interval setting gas inlet in communication with the oxygen supply end, an inhalation interval setting gas inlet in communication with the oxygen supply end, an intermittent oxygen output outlet in communication with the pilot-operated cylinder, an exhalation time adjusting button, and an inhalation time adjusting button. The timing valve module receives oxygen from the oxygen supply end through the exhalation interval setting gas inlet and the inhalation interval setting gas inlet and is driven by the oxygen thus received. Both the exhalation time adjusting button and the inhalation time adjusting button can be adjusted to control the timing at which the exhalation interval setting gas inlet or the inhalation interval setting gas inlet outputs oxygen via the intermittent oxygen output outlet and thereby drives the pilot-operated cylinder to drive the gas-operated valve.

In addition to the main structural features described above, the timing valve module includes a first gas valve set and a second gas valve set. Each of the first gas valve set and the second gas valve set has a throttle valve and a 2-position 3-way directional valve in communication with the throttle valve. The 2-position 3-way directional valve of the first gas valve set is in communication with the exhalation interval setting gas inlet, the intermittent oxygen output outlet, and the throttle valve of the second gas valve set. The 2-position 3-way directional valve of the second gas valve set is in communication with the inhalation interval setting gas inlet and the throttle valve of the first gas valve set. The throttle valve of the first gas valve set can be adjusted via the exhalation time adjusting button, and the throttle valve of the second gas valve set can be adjusted via the inhalation time adjusting button.

In addition to the main structural features described above, the gas-operated valve is in communication with a tidal-volume gas output end configured for outputting oxygen intermittently, a tidal-volume gas flow rate adjusting valve is in communication between the gas-operated valve and the tidal-volume gas output end, and an oxygen input end is in communication between the oxygen supply end and the gas-operated valve of the oxygen supply valve unit.

In addition to the main structural features described above, the oxygen input end is in communication with an intermittent mandatory ventilation (IMV) output end configured for mandatory continuous oxygen output, and an IMV flow rate adjusting valve is in communication between the oxygen input end and the IMV output end. The IMV output end supplies oxygen to a patient through an external breathing bag.

In addition to the main structural features described above, the oxygen input end is in communication with a manual oxygen supply button valve configured for manual oxygen output so that oxygen can be manually supplied to the tidal-volume gas output end and an exhalation valve driving gas output end.

In addition to the main structural features described above, an adapter unit is provided which has a tidal-volume gas connection/input hole in communication with the tidal-volume gas output end, a tidal-volume gas connection/output hole in communication with the tidal-volume gas connection/input hole, a pressure relief valve in communication with the tidal-volume gas connection/input hole and the tidal-volume gas connection/output hole, an exhalation valve driving gas connection/input hole in communication with the exhalation valve driving gas output end, an exhalation valve driving gas connection/output hole in communication with the exhalation valve driving gas connection/input hole, and an oxygen supply pressure gage. The tidal-volume gas connection/output hole is configured for supplying oxygen to a patient. The oxygen supply pressure gage is, in the vicinity of a patient, brought into communication with the tidal-volume gas connection/output hole so as to show the pressure of the oxygen supplied.

In addition to the main structural features described above, a ratio control valve and a shuttle valve are provided. The ratio control valve is in communication between the gas-operated valve and the oxygen supply end. The shuttle valve is in communication between the oxygen supply end and an external hyperbaric chamber gas supply end. The shuttle valve and the ratio control valve are linked to each other. The shuttle valve is driven by the pressure at the oxygen supply end and the pressure at the hyperbaric chamber gas supply end respectively and, in turn, drives the ratio control valve to adjust the pressure at which the oxygen supply end supplies oxygen to the gas-operated valve.

In addition to the main structural features described above, an operating pressure adjusting valve through which a basic operating output pressure can be set is in communication between the oxygen supply end and the shuttle valve, and the oxygen supply end is in communication with the exhalation interval setting gas inlet and the inhalation interval setting gas inlet via a pressure regulating valve through which a pressure can be set.

In addition to the main structural features described above, the shuttle valve and the hyperbaric chamber gas supply end are in communication with each other via a chamber pressure input end. The chamber pressure input end is in communication with a chamber pressure gage. Besides, an operating pressure gage is in communication between the ratio control valve and the gas-operated valve, and the pressure regulating vale is in communication with a timing valve pressure gage.

### BRIEF DESCRIPTION OF THE SEVERAL VIEWS OF THE DRAWINGS

The structure as well as a preferred mode of use, further objects, and advantages of the present invention will be best understood by referring to the following detailed description of some illustrative embodiments in conjunction with the accompanying drawings, in which:

FIG. 1 is a schematic drawing showing the arrangement of the elements of a preferred embodiment of the present invention;

FIG. 2 is an assembled perspective view of the embodiment shown in FIG. 1;

FIG. 3 is a pneumatic circuit diagram of the embodiment shown in FIG. 1;

FIG. 4 is another assembled perspective view of the embodiment shown in FIG. 1 and is taken from a different viewpoint from that of FIG. 2;

FIG. 5 is a perspective view of accessories for use in the embodiment shown in FIG. 1;

FIG. 6 shows a state of use following that shown in FIG. 3; and

FIG. 7 shows a state of use following that shown in FIG. 6.

### DETAILED DESCRIPTION OF THE INVENTION

Please refer to FIG. 1 for a schematic drawing showing the arrangement of the elements of a preferred embodiment of the present invention; FIG. 2 for an assembled perspective view of the embodiment shown in FIG. 1; FIG. 3 for a pneumatic circuit diagram of the embodiment shown in FIG. 1; FIG. 4 for another assembled perspective view of the embodiment shown in FIG. 1, taken from a different viewpoint from that of FIG. 2; FIG. 5 for a perspective view of accessories for use in the embodiment shown in FIG. 1; FIG. 6 for a state of use following that shown in FIG. 3; and FIG. 7 for a state of use following that shown in FIG. 6.

Referring to FIG. 1 to FIG. 7, the present invention provides an oxygen-supplying respirator requiring no electric power, wherein the oxygen-supplying respirator includes an oxygen supply valve unit 2 in communication with an external oxygen supply end 8. The oxygen supply valve unit 2 is configured for turning on and off oxygen output from the oxygen supply end 8. In practice, the oxygen supply end 8 may be an oxygen cylinder.

An inhalation/exhalation time setting valve unit 3 is in communication between the oxygen supply valve unit 2 and the oxygen supply end 8. The inhalation/exhalation time setting valve unit 3 receives the oxygen continuously supplied from the oxygen supply end 8 and outputs oxygen intermittently to the oxygen supply valve unit 2. The intermittently output oxygen drives the oxygen supply valve unit 2 to turn on the oxygen supply end 8 intermittently, thereby enabling intermittent output of oxygen.

More specifically, the present invention further includes the following technical features:

The oxygen supply valve unit 2 includes a pilot-operated cylinder 22 and a gas-operated valve 21 to be driven by the pilot-operated cylinder 22 in order to turn on or off oxygen output from the oxygen supply end 8 to a patient. The inhalation/exhalation time setting valve unit 3, on the other hand, includes a timing valve module 30. The timing valve module has an exhalation interval setting gas inlet 31 in communication with the oxygen supply end 8, an inhalation interval setting gas inlet 32 in communication with the oxygen supply end 8, an intermittent oxygen output outlet 33 in communication with the pilot-operated cylinder 22, an exhalation time adjusting button 34, and an inhalation time adjusting button 35. The timing valve module 30 receives oxygen from the oxygen supply end 8 through the exhalation interval setting gas inlet 31 and the inhalation interval setting gas inlet 32 and is driven by the oxygen received. The exhalation time adjusting button 34 and the inhalation time adjusting button 35 can be adjusted by a user so as to control the timing at which the exhalation interval setting gas inlet 31 or the inhalation interval setting gas inlet 32 outputs oxygen via the intermittent oxygen output outlet 33 and thereby drives the pilot-operated cylinder 22 to drive the gas-operated valve 21.

By adjusting the inhalation time adjusting button 35, the time for which oxygen is allowed to flow into a patient's breathing tube circuit can be increased or decreased. By adjusting the exhalation time adjusting button 34, the time for which oxygen is stopped from flowing into a patient's breathing tube circuit can be increased or decreased.

The timing valve module 30 includes a first gas valve set 36 and a second gas valve set 37. The first gas valve set 36 has a throttle valve 361 and a 2-position 3-way directional valve 362. Similarly, the second gas valve set 37 has a throttle valve 371 and a 2-position 3-way directional valve 372. The 2-position 3-way directional valve 362 of the first gas valve set 36 is in communication with the exhalation interval setting gas inlet 31, the intermittent oxygen output outlet 33, and the throttle valve 371 of the second gas valve set 37. The 2-position 3-way directional valve 372 of the second gas valve set 37 is in communication with the inhalation interval setting gas inlet 32 and the throttle valve 361 of the first gas valve set 36. The throttle valve 361 of the first gas valve set 36 is linked to and adjustable by the exhalation time adjusting button 34. The throttle valve 371 of the second gas valve set 37 is linked to and adjustable by the inhalation time adjusting button 35.

The gas-operated valve 21 is in communication with a tidal-volume gas output end 41 by way of a first quick coupling 11, wherein the tidal-volume gas output end 41 is configured for outputting oxygen intermittently and the first quick coupling 11 may be a 4-way coupling. In addition, a tidal-volume gas flow rate adjusting valve 51 is in communication between the gas-operated valve 21 and the tidal-volume gas output end 41. In fact, the tidal-volume gas flow rate adjusting valve 51 is in communication between the first quick coupling 11 and the tidal-volume gas output end 41.

By adjusting the tidal-volume gas flow rate adjusting valve 51, the amount of gas to be delivered to a patient's breathing tube circuit in each inhalation stage can be increased or decreased.

An oxygen input end 40 is in communication between the oxygen supply end 8 and the gas-operated valve 21 of the oxygen supply valve unit 2. More specifically, the oxygen input end 40 is in communication with a second quick coupling 12, which may be a 5-way coupling. Additionally, a toggle valve 15 is in communication between the oxygen input end 40 and the second quick coupling 12. The toggle valve 15 serves to turn on and off oxygen input to the second quick coupling 12 from the oxygen input end 40.

The oxygen input end 40 is in communication with an intermittent mandatory ventilation (IMV) output end 42 via the toggle valve 15 and the second quick coupling 12, wherein the IMV output end 42 is configured for outputting oxygen in a mandatory and continuous manner. Also, an IMV flow rate adjusting valve 52 is in communication between the oxygen input end 40 and the IMV output end 42. In fact, the IMV flow rate adjusting valve 52 is in communication between the second quick coupling 12 and the IMV output end 42. The IMV output end 42 supplies oxygen to a patient's breathing tube circuit through an external IMV breathing bag.

The oxygen input end 40 is further in communication with an exhalation valve driving gas output end 43 by way of the toggle valve 15, the second quick coupling 12, and the first quick coupling 11, wherein the exhalation valve driving gas output end 43 is configured for outputting oxygen manually. A manual oxygen supply button valve 53 is in communication between the oxygen input end 40 and the exhalation valve driving gas output end 43. In fact, the manual oxygen supply button valve 53 is in communication between the second quick coupling 12 and the first quick coupling 11. By pressing the manual oxygen supply button valve 53, a continuous oxygen flow is allowed, and this oxygen flow can be used to supply oxygen to a patient whenever the operator desires.

The oxygen-supplying respirator of the present invention further includes a ratio control valve 23 in communication between the gas-operated valve 21 and the oxygen supply end 8, and a shuttle valve 24 in communication between the oxygen supply end 8 and a hyperbaric chamber gas supply end 9 of an external hyperbaric chamber. The shuttle valve 24 and the ratio control valve 23 are linked to each other. The shuttle valve 24 is driven by the gas pressure at the oxygen supply end 8 and the gas pressure at the hyperbaric chamber gas supply end 9 respectively and hence drives the ratio control valve 23 to adjust the pressure at which the oxygen supply end 8 supplies oxygen to the gas-operated valve 21.

An operating pressure adjusting valve 54 is in communication between the oxygen supply end 8 and the shuttle valve 24 so that a basic operating output pressure can be set through the operating pressure adjusting valve 54. Further, the oxygen supply end 8 is in communication with the exhalation interval setting gas inlet 31 and the inhalation interval setting gas inlet 32 via a pressure regulating valve 55 through which a pressure can be set. With the pressure adjusting valve 54, a basic operating output pressure of 15 psi can be set to automatically increase gas flow and thereby compensate for chamber pressure variations.

The oxygen supply end 8 is in communication with the pressure adjusting valve 54 and the pressure regulating valve 40 via the oxygen input end 40, the toggle valve 15, and the second quick coupling 12. The pressure regulating valve 55 is in communication with the exhalation interval setting gas inlet 31 and the inhalation interval setting gas inlet 32 via a third quick coupling 13, which may be a 4-way coupling.

The shuttle valve 24 is in communication with the hyperbaric chamber gas supply end 9 via a chamber pressure input end 44. The ratio control valve 23 and the gas-operated valve 21 are in communication with each other and with an operating pressure gage 61 via a fourth quick coupling 14. Moreover, the pressure regulating valve 55 is in communication with a timing valve pressure gage 62 through the third quick coupling 13, and the chamber pressure input end 44 is in communication with a chamber pressure gage 63. The operating pressure gage 61 shows the pressure of the gas output from the operating pressure adjusting valve 54. The timing valve pressure gage 62 shows the preset pressure (typically 50 psi) of the timing valve module 30 configured for controlling inhalation and exhalation. The chamber pressure gage 63 shows the actual pressure of the external hyperbaric chamber.

The oxygen supply end 8 is in communication with the gas-operated valve 21 of the oxygen supply valve unit 2 via the oxygen input end 40, the toggle valve 15, the ratio control valve 23, and the fourth quick coupling 14.

The oxygen-supplying respirator of the present invention further includes an adapter unit 7. The adapter unit 7 is provided with a tidal-volume gas connection/input hole 71 in communication with the tidal-volume gas output end 41, a tidal-volume gas connection/output hole 72 in communication with the tidal-volume gas connection/input hole 71, a pressure relief valve 73 in communication with the tidal-volume gas connection/input hole 71 and the tidal-volume gas connection/output hole 72, an exhalation valve driving gas connection/input hole 74 in communication with the exhalation valve driving gas output end 43, an exhalation valve driving gas connection/output hole 75 in communication with the exhalation valve driving gas connection/input hole 74, and an oxygen supply pressure gage 76.

The tidal-volume gas connection/output hole 72 is configured for supplying oxygen to a patient. The adapter unit 7 has a pressure gage gas-guiding hole 761 in communication with the oxygen supply pressure gage 76, and the oxygen supply pressure gage 76 is, in the vicinity of a patient, brought into communication with the tidal-volume gas connection/output hole 72 via the pressure gage gas-guiding hole 761 so as to show the actual pressure (cm H₂O) of the oxygen supplied to the patient's breathing tube circuit. By adjusting the pressure relief valve 73, gas pressure can be regulated to the highest doctor-prescribed pressure, as shown by the oxygen supply pressure gage 76.

By adjusting the IMV flow rate adjusting valve 52, the amount of oxygen to be supplied to an IMV breathing bag can be increased or decreased. The IMV breathing bag is located at one end of the adapter unit 7 and is linearly connected to a patient's breathing tube circuit. The IMV flow rate adjusting valve 52 and the IMV output end 42 are so designed that a patient is allowed to breathe spontaneously in between the mechanical respiratory actions of the respirator.

The oxygen-supplying respirator of the present invention further includes a respirator housing 1. Provided in the housing 1 are the first, second, third, and fourth quick couplings 11, 12, 13 and 14; the gas-operated valve 21, the pilot-operated cylinder 22, the ratio control valve 23, and the shuttle valve 24 of the oxygen supply valve unit 2; the timing valve module 30, the exhalation interval setting gas inlet 31, the inhalation interval setting gas inlet 32, and the intermittent oxygen output outlet 33 of the inhalation/exhalation time setting valve unit 3; the throttle valves 361 and 371 and the 2-position 3-way directional valves 362 and 372 of the first and second gas valve sets 36 and 37; and the pressure regulating valve 55. Provided on the outer walls of the housing 1 are the toggle valve 15, the exhalation time adjusting button 34, the inhalation time adjusting button 35, the oxygen input end 40, the tidal-volume gas output end 41, the IMV output end 42, the exhalation valve driving gas output end 43, the chamber pressure input end 44, the tidal-volume gas flow rate adjusting valve 51, the IMV flow rate adjusting valve 52, the manual oxygen supply button valve 53, the pressure adjusting valve 54, the operating pressure gage 61, the timing valve pressure gage 62, and the chamber pressure gage 63.

The present invention can be implemented using the elements described above. The respirator of the present invention is suitable for use in a hyperbaric chamber as well as at normal ambient pressure. Principally, the respirator works by outputting oxygen from the tidal-volume gas output end 41 to a patient's breathing tube circuit through the tidal-volume gas connection/output hole 72 of the adapter unit 7. To begin with, the oxygen input end 40 is brought into communication with an oxygen cylinder functioning as the oxygen supply end 8, and the exhalation time and the inhalation time can be adjusted by means of the exhalation time adjusting button 34 and the inhalation time adjusting button 35 respectively. Afterward, the tidal-volume gas connection/output hole 72 is brought into communication with the patient's breathing tube circuit, so as for the oxygen supply end 8 to input oxygen into the respirator through the oxygen input end 40 and thereby drive the respirator into operation.

The oxygen input through the oxygen input end 40 flows through the toggle valve 15 to the ratio control valve 23 and the second quick coupling 12. The oxygen flowing to the second quick coupling 12 continues on to the pressure adjusting valve 54 and then to the shuttle valve 24. If the respirator is used in a hyperbaric chamber, the chamber pressure input end 44 receives the pressure of the hyperbaric chamber through the hyperbaric chamber gas supply end 9. If, in that case, the pressure is greater than 15 psi, the ratio control valve 23 will pressurize proportionally during oxygen output in order to supply oxygen stably; otherwise, the flow rate of the oxygen supplied will be reduced under the pressure of the hyperbaric chamber. The pressure at the chamber pressure input end 44 will be shown by the chamber pressure gage 63.

The oxygen flowing to the second quick coupling 12 also flows to the pressure regulating valve 55 so that its pressure can be set. Oxygen then flows from the pressing regulating valve 55 to the third quick coupling 13, where the oxygen is guided into the exhalation interval setting gas inlet 31, the inhalation interval setting gas inlet 32, and the timing valve pressure gage 62 respectively.

The oxygen flowing to the inhalation interval setting gas inlet 32 runs through the 2-position 3-way directional valve 372 of the second gas valve set 37 to the throttle valve 361 of the first gas valve set 36, is delayed by the throttle valve 361 of the first gas valve set 36, and then drives the 2-position 3-way directional valve 362 of the first gas valve set 36. Consequently, the oxygen flowing to the exhalation interval setting gas inlet 31 passes through the 2-position 3-way directional valve 362 of the first gas valve set 36 and reaches the intermittent oxygen output outlet 33 and the throttle valve 371 of the second gas valve set 37. The oxygen reaching the throttle valve 371 is delayed by the throttle valve 371 and then drives the 2-position 3-way directional valve 372 of the second gas valve set 37 such that the oxygen supplied to the inhalation interval setting gas inlet 32 is blocked from flowing to the throttle valve 361 of the first gas valve set 36.

Thus, the delaying effects of the throttle valves 361 and 371 on the 2-position 3-way directional valves 362 and 372 control the time at which and the frequency with which oxygen is output through the intermittent oxygen output outlet 33. Further, the time for which the 2-position 3-way directional valves 362 and 372 are respectively delayed by the throttle valves 361 and 371 can be adjusted via the exhalation time adjusting button 34 and the inhalation time adjusting button 35.

The oxygen output from the intermittent oxygen output outlet 33 flows to the pilot-operated cylinder 22, causing the pilot-operated cylinder 22 to drive the gas-operated valve 21. At the same time, the oxygen flowing to the ratio control valve 23 runs through the fourth quick coupling 14 to the gas-operated valve 21. The oxygen running through the fourth quick coupling 14 goes also to the operating pressure gage 61, so as for the operating pressure gage 61 to show the pressure of the output gas. As such, the pilot-operated cylinder 22 drives the gas-operated valve 21 to intermittently allow the oxygen flowing to the ratio control valve 23 to pass through the first quick coupling 21, the tidal-volume gas flow rate adjusting valve 51, and the tidal-volume gas output end 41 and be output intermittently from the tidal-volume gas output end 41. The oxygen intermittently output from the tidal-volume gas output end 41 will be input into the patient's breathing tube circuit by way of the tidal-volume gas connection/input hole 71 and the tidal-volume gas connection/output hole 72 of the adapter unit 7.

Alternatively, oxygen may be supplied to the patient's breathing tube circuit via the IMV output end 42 and an IMV breathing bag connected thereto. In that case, oxygen is supplied to the IMV breathing bag in a forced and continuous manner while the IMV breathing bag allows the patient to breathe spontaneously in between the mechanical respiratory actions of the respirator.

It is also feasible to supply oxygen to a patient's breathing tube circuit manually. For example, should the inhalation/exhalation time setting valve unit 3 of the respirator fail, the manual oxygen supply button valve 53 can be manually pressed to output oxygen to a patient's breathing tube circuit through the tidal-volume gas connection/input hole 71 and the tidal-volume gas connection/output hole 72 of the adapter unit 7. Nevertheless, whether oxygen is delivered manually or is automatically output in a timed manner, there will always be gas output from the exhalation valve driving gas output end 43. The gas output from the exhalation valve driving gas output end 43 is intended to shut an external exhalation valve when oxygen is supplied to the patient for inhalation, and once the oxygen output is stopped (i.e., in an exhalation stage), the gas output from the exhalation valve driving gas output end 43 will open the external exhalation valve to discharge the gas exhaled from the patient. The aforesaid process repeats itself to control the patient's tidal volume at a fixed level.

According to the above, the oxygen supply end 8 can be used to supply oxygen to the inhalation/exhalation time setting valve unit 3 and the oxygen supply valve unit 2 continuously, thereby driving the inhalation/exhalation time setting valve unit 3 to output oxygen intermittently to the oxygen supply valve unit 2. The oxygen supply valve unit 2 will be driven by the intermittently output oxygen and turn on the oxygen supply end 8 intermittently in response, so that oxygen can be output to a patient in an intermittent fashion.

In a nutshell, the respirator of the present invention does not require electric power as its driving force. Rather, the pressure of the oxygen output from an oxygen cylinder can be used to drive the respirator into operation and control the flow rate of its oxygen output. Thus, the object of driving the respirator not by electricity but by the oxygen supply pressure at an oxygen supply end is achieved.

What is claimed is:

1. An oxygen-supplying respirator requiring no electric power, comprising an oxygen supply valve unit which is in communication with an external oxygen supply end and which is configured for turning on and off oxygen output from the oxygen supply end, the oxygen-supplying respirator being characterized in that:
an inhalation/exhalation time setting valve unit is in communication between the oxygen supply valve unit and the oxygen supply end, wherein the inhalation/exhalation time setting valve unit receives oxygen supplied continuously from the oxygen supply end and outputs oxygen to the oxygen supply valve unit intermittently, thereby driving the oxygen supply valve unit to turn on the oxygen supply end intermittently in order to output oxygen intermittently.

2. The oxygen-supplying respirator of claim 1, wherein the oxygen supply valve unit comprises a pilot-operated cylinder and a gas-operated valve, the gas-operated valve being driven by the pilot-operated cylinder to turn on or off oxygen output from the oxygen supply end to a patient; and wherein the inhalation/exhalation time setting valve unit comprises a timing valve module, the timing valve module having an exhalation interval setting gas inlet in communication with the oxygen supply end, an inhalation interval setting gas inlet in communication with the oxygen supply end, an intermittent oxygen output outlet in communication with the pilot-operated cylinder, an exhalation time adjusting button, and an inhalation time adjusting button, the timing valve module receiving oxygen from the oxygen supply end through the exhalation interval setting gas inlet and the inhalation interval setting gas inlet and being driven by the oxygen thus received, the exhalation time adjusting button and the inhalation time adjusting button being adjustable to control a timing at which the exhalation interval setting gas inlet or the inhalation interval setting gas inlet outputs oxygen via the intermittent oxygen output outlet and thereby drives the pilot-operated cylinder to drive the gas-operated valve.

3. The oxygen-supplying respirator of claim 2, wherein the timing valve module comprises a first gas valve set and a second gas valve set, each said gas valve set having a throttle valve and a 2-position 3-way directional valve in communication with the throttle valve, the 2-position 3-way directional valve of the first gas valve set being in communication with the exhalation interval setting gas inlet, the intermittent oxygen output outlet, and the throttle valve of the second gas valve set; the 2-position 3-way directional valve of the second gas valve set being in communication with the inhalation interval setting gas inlet and the throttle valve of the first gas valve set; the throttle valve of the first gas valve set being adjustable by the exhalation time adjusting button, the throttle valve of the second gas valve set being adjustable by the inhalation time adjusting button.

4. The oxygen-supplying respirator of claim 2, wherein the gas-operated valve is in communication with a tidal-volume gas output end configured for outputting oxygen intermittently; a tidal-volume gas flow rate adjusting valve is in communication between the gas-operated valve and the tidal-volume gas output end; and an oxygen input end is in communication between the oxygen supply end and the gas-operated valve of the oxygen supply valve unit.

5. The oxygen-supplying respirator of claim 4, wherein the oxygen input end is in communication with an intermittent mandatory ventilation (IMV) output end configured for mandatory continuous oxygen output, and an IMV flow rate adjusting valve is in communication between the oxygen input end and the IMV output end, the IMV output end supplying oxygen to a patient through an external breathing bag.

6. The oxygen-supplying respirator of claim 4, wherein the oxygen input end is in communication with a manual oxygen supply button valve configured for manual oxygen output so that oxygen can be manually supplied to the tidal-volume gas output end and an exhalation valve driving gas output end.

7. The oxygen-supplying respirator of claim 6, further comprising an adapter unit, the adapter unit having a tidal-volume gas connection/input hole in communication with the tidal-volume gas output end, a tidal-volume gas connection/output hole in communication with the tidal-volume gas connection/input hole, a pressure relief valve in communication with the tidal-volume gas connection/input hole and the tidal-volume gas connection/output hole, an exhalation valve driving gas connection/input hole in communication with the exhalation valve driving gas output end, an exhalation valve driving gas connection/output hole in communication with the exhalation valve driving gas connection/input hole, and an oxygen supply pressure gage, the tidal-volume gas connection/output hole being configured for supplying oxygen to a patient, the oxygen supply pressure gage being brought into communication with the tidal-volume gas connection/output hole, in a vicinity of the patient, so as to show a pressure of the oxygen supplied.

8. The oxygen-supplying respirator of claim 2, further comprising a ratio control valve and a shuttle valve, the ratio control valve being in communication between the gas-operated valve and the oxygen supply end, the shuttle valve being in communication between the oxygen supply end and an external hyperbaric chamber gas supply end, the shuttle valve and the ratio control valve being linked to each other, wherein the shuttle valve is driven by a pressure at the oxygen supply end and a pressure at the hyperbaric chamber gas supply end respectively and, in turn, drives the ratio control valve to adjust a pressure at which the oxygen supply end supplies oxygen to the gas-operated valve.

9. The oxygen-supplying respirator of claim 8, wherein an operating pressure adjusting valve through which a basic operating output pressure can be set is in communication between the oxygen supply end and the shuttle valve, and the oxygen supply end is in communication with the exhalation interval setting gas inlet and the inhalation interval setting gas inlet via a pressure regulating valve through which a pressure can be set.

10. The oxygen-supplying respirator of claim 9, wherein the shuttle valve and the hyperbaric chamber gas supply end are in communication with each other through a chamber pressure input end; the chamber pressure input end is in communication with a chamber pressure gage; an operating pressure gage is in communication between the ratio control valve and the gas-operated valve; and the pressure regulating vale is in communication with a timing valve pressure gage.
